(19) United States
(12) Patent Application Publication
(10) Pub. No.: US 2025/0099016 A1
(43) Pub. Date: Mar. 27, 2025

(54) SYSTEMS AND METHODS FOR ESTIMATING INTEROCEPTIVE AWARENESS STATE USING EYE-TRACKING MEASUREMENTS

(71) Applicant: NEW YORK UNIVERSITY, New York, NY (US)

(72) Inventors: Rose T. Faghih, New York, NY (US); Saman Khazaei, Brooklyn, NY (US)

(73) Assignee: NEW YORK UNIVERSITY, New York, NY (US)

(21) Appl. No.: 18/892,100

(22) Filed: Sep. 20, 2024

Related U.S. Application Data
(60) Provisional application No. 63/584,814, filed on Sep. 22, 2023.

(51) Int. Cl. A61B 5/00; A61B 5/16

(52) U.S. Cl. CPC A61B 5/4064; A61B 5/163; A61B 5/725; A61B 5/7435

(57) ABSTRACT

Systems and methods for continuously estimating interoceptive awareness. An eye tracker device collects eye-tracking data comprising pupillometry measurements and gaze measurements. A computer system processes the eye-tracking data, with an extraction module and an interoceptive awareness state estimator to produce an estimated interoceptive awareness state shown on a display of the computer system.

### CROSS-REFERENCE TO RELATED PATENT APPLICATIONS

This application claims the benefit of and priority to U.S. Provisional Application No. 63/584,814, filed on Sep. 22, 2023, the entire disclosure of which is hereby incorporated by reference herein.

### STATEMENT OF GOVERNMENT INTEREST

This invention was made with government support under 2226123 awarded by the National Science Foundation and R35 GM151353 awarded by the National Institutes of Health. The government has certain rights in the invention.

### TECHNICAL FIELD

The present disclosure relates generally to systems and methods for estimating interoceptive awareness state using eye-tracking measurements.

### BACKGROUND

Human-machine interfaces, in particular brain-machine interfaces (“BCI”) as becoming more important as computer technology is integrated into society and daily human activities. A major component for human-machine interfacing relates to tracking, interpreting, predicting, and/or replicating human eye movement and function. While some advances have been made with regard to the physical tracking of the eye, such as retina position, eye movement, and the like, the less directly observable aspects of the eye, in particular communication between the eye and the brain, are less understood in the context of a human-machine interface.

The anatomical structure and complex function make the eyes a difficult human component for developing human-machine interfaces. One of the essential tissues of the eye is the retina which is a thin layer located near the optic nerve in the back of the eye; it is mostly described as an extension of the central nervous system. In the very center of the retina, there is a part called the fovea. The eye-mind link assumption expresses that there is enough motivation to move the eye and fixate it so that the fovea is pointed at the potential stimulus and the object of interest. Although there is no solid conclusion regarding the link between the eye and mind yet, the association between eye movements and the internal brain states is undeniable.

The awareness state of sensory information can be defined as interoceptive awareness (“IA”) state. In other words, IA conveys the brain's interpretation of the bodily sensation in response to an external stimulus. Hence, the internal IA brain state is positively associated with attention and cognitive arousal in relation to the experienced stimuli. The IA state is depicted as an internal brain state and indicative of one's emotional awareness and arousal. Hence, arousal events may be used to decode the underlying interoception. From the Neurophysiology perspective, arousal may refer to the degree of wakefulness, the state of being conscious, various levels of awareness, and the intensity of emotions. Systems and methods herein seek to decode the underlying interoceptive awareness state from the physiological responses of the eye in the presence of arousing stimuli.

Similar to most of the body's muscle movements, for moving the eye, it is required to contract one muscle and relax its opponent. Eye position and orientation are controlled by six extraocular muscles (3 pairs). All of these movements are governed by the central nervous system. On the other hand, the pupil size of the eye can be adjusted by the variations of light levels as well as cognitive factors and autonomic arousal. Two muscles are involved in changing the pupil diameter: the dilator pupillae which dilates the pupil and the sphincter pupillae that constricts the pupil. The dilator pupillae is controlled by the sympathetic nervous system; the sphincter pupillae is governed by the parasympathetic nervous system which plays a crucial role in arousal regulation.

Multiple studies have investigated the feasibility of inferring arousal level from the eye-tracking measurement: The correlation between the fixation duration and arousal has been illustrated such that a longer fixation duration on stimulus was followed by an increase in arousal level. The saccadic velocity and arousal relationship is another aspect that has been widely researched. The impacts of arousal on saccadic movements may appear on the level of the excitatory burst neurons such that the firing rates encode the velocity signal of saccades. It has been shown that reduced arousal results in decreased saccadic velocity while increased arousal may increase the saccadic velocity. The arousing events are not the only factors that contribute to changes in autonomic nervous system (“ANS”) activity and pupil size. Given the association between the arousal and IA, it has been noted that interoception also evokes the ANS activation. Hence, observing the process from the control theory lens, the pupil size signal can be seen as the informative metrics of artificial intelligence (“AI”) in response to arousal events. Additionally, several studies have illustrated that pupil dilation is associated with an increase in the arousal level while arousal reduction is followed by pupil reduction constriction.

Thus far, most of the illustrated metrics have been employed in heterogeneous paradigms, and studies mainly consider either pupillometry or eye movements to decode the internal state, solely. A limited number of studies have applied more than one eye-tracking feature thus far. There is a lack of systematic approach to quantify the interoceptive awareness considering the pupillometry and eye gaze measurements, simultaneously. Additionally, the majority of proposed brain state decoders apply machine learning (“ML”) and deep learning (“DL”) algorithms which require a training session beforehand. For the experiments described herein, both pupillometry and eye gaze were employed to recover the arousal-related neural activities (i.e., arousal events) in a near real-time manner. The underlying IA state is then decoded during the fear conditioning experiment (Pavlovian experiments) and with no prior training session. The fear conditioning experiment is a behavioral experiment that subjects learn to predict the aversive events throughout the experiment. Different types of neutral cues (conditioned stimuli (“CS”)) can be paired with the aversive events (unconditioned stimuli (“US”)). In this particular experiment, two types of CS have been used: CS+ that will be accompanied by US 50% of the time; and CS− which will not be accompanied by US at all (US−).

It is believed that pupil size, pupillary response, fixation duration, saccadic velocity, and saccade peaks are associated with IA. The binary-type arousal-related neural activities are recovered and a binary decoder is employed to decode the IA state in a non-invasive manner. FIG. 4 outlines an overview of the proposed framework. The eye-based findings are evaluated by considering electrodermal activity (“EDA”) as a physiological metric of arousal which is positively correlated with interoceptive awareness. Therefore, the EDA-based arousal decoder is employed and quantify the arousal level. The experimental results indicate an observed agreement between the eye-based IA state and EDA-based arousal levels.

Since the IA state is a function of underlying arousal level, the system recovers the arousal-related neural activities (arousal event) and quantifies the person-specific interoceptive awareness, continuously. This can address the existing challenges in the biofeedback design paradigm for mental health and cognitive disorders (such as depression, post-traumatic stress disorder (“PTSD”), Alzheimer's disease, and attention deficit disorder); and it would transform the mental disorder treatments to a new level.

Particularly, to design the optimal, adaptive, and person-specific biofeedback based on human/AI interactions, there is a demand to complement the exteroceptive sensing of users' interactions with interoceptive sensing states such as arousal state. Hence, developing an interpretable mathematical model of continuous real-time interoceptive awareness using physiological signals-such as eye-tracking information-would enable real-time interoceptive state modulation via human/AI interaction.

In comparison with the other physiological signals that common wearables provide, eye-tracking measurements offer fewer artifacts by preserving the subject's internal state baseline. Particularly, there would be no sensor attachment to the subject, and the data can be collected without distortion of the subject's awareness, in the absence of distraction, and out of a controlled laboratory environment. Additionally, monitoring the IA state through the eyes is applicable to everyday life settings, and the sensors can be implemented in smartphones, laptops, cars, and any surface. Hence, the implementation can go beyond the clinical settings, and it can be installed in classrooms and smart offices.

Additionally, eye-tracking metrics have been employed in heterogenous paradigms, and most of the studies have investigated either pupillometry or eye movements, solely. A limited number of studies have applied more than one eye-tracking feature thus far. Hence, there is a lack of systematic approach to quantify the interoceptive awareness considering the pupillometry and eye gaze measurements, simultaneously. The systems and methods described herein empower us to continuously monitor the underlying interoceptive awareness state through the eye while most of the existing approaches can only map the non-continuous interoceptive awareness (self-report).

### SUMMARY

Described herein are systems and methods for estimating interoceptive awareness (“IA”) state using eye-tracking measurements. The awareness state of sensory information can be defined as an IA state. That is, as used herein, IA conveys the brain interpretation of the bodily sensation in response to an external stimulus. Hence, the internal IA brain state is positively associated with attention and cognitive arousal in relation to the observed stimuli.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

### BRIEF DESCRIPTION OF THE FIGURES

The foregoing and other features of the present disclosure will become more fully apparent from the following description and appended claims, taken in conjunction with the accompanying drawings. Understanding that these drawings depict only several implementations in accordance with the disclosure and are therefore not to be considered limiting of its scope, the disclosure will be described with additional specificity and detail through use of the accompanying drawings.

FIG. 1 is an illustration of a system for estimation of an interoceptive awareness (“IA”) state from the eye-tracking measurements in accordance with the present disclosure.

FIG. 2A is a block diagram of the system implementation of the system shown in FIG. 1. FIG. 2B is another block diagram of the system implementation of the system shown in FIG. 1.

FIG. 3 is an overview of the IA state estimation from the eye movements and pupillometry measurements. This diagram depicts an overview of the proposed framework given the eye movements and pupillometry measurements as the input of the system, and estimated IA state as the main output, according to an example embodiment.

FIG. 4 is a flow diagram illustrating a method of estimating an IA state from the eye movements and pupillometry measurements.

FIG. 5 an illustration of an exemplary operating environment.

FIG. 6 is a block diagram of a hardware configuration for the exemplary operating environment shown in FIG. 5.

FIGS. 7A-7B are examples of recovered arousal-related autonomic nervous system (“ANS”) activation from skin conductance. FIG. 7A represents the complete recorded data, and FIG. 7B shows a random window of the experiment. Sub-panel A shows raw skin conductance signal. Sub-panel B shows recovered arousal-related neural stimuli due to ANS activation. The red background color corresponds to the CS+US+ trials; the yellow background color stands for CS+US− trials; and the blue one indicates the CS− trials. Black dashed lines mark the moment that electric shocks exist.

FIGS. 8A-8B are examples of eye-based feature extraction steps in recovering the arousal events and forming the observation vector. FIG. 8A represents the complete recorded data, and FIG. 8B shows a random window of the experiment. Sub-panel A shows the occurrence of the fixation events (blue impulses), and the fixation duration (the amplitude of each impulse). Sub-panel B shows the average pupil size (derived from both eye). Sub-panel C shows the pupil size derivative (dilation and constriction) of the subject. Sub-panel D shows the average velocity (derived from both eye). Sub-panel E shows the acceleration (derived from both eye). Sub-panel F shows the recovered arousal-related events from the eye that form a binary-type observation vector (blue impulses). The black dash line stands for the applied threshold in each step; At the top of each sub-panel, green color depicts the bin's location where the threshold is reached. The red background color corresponds to the CS+US+ trials; the yellow background color stands for CS+US− trials; and the blue one indicates the CS− trials.

FIGS. 9A-9D show the event related potential-like (“ERP”-like) analysis in the fear conditioning experiment. FIG. 9A shows the average of pupil size across CS+US+ trials (red), CS+US− trials (yellow), and CS− trials (blue) and its 95% confidence limits. FIG. 9B shows the average velocity of both eyes (left and right) across CS+US+ trials (red), CS+US− trials (yellow), and CS− trials (blue) and its 95% confidence limits. FIG. 9C shows the average of decoded IA state from eye-tracking across CS+US+ trials (red), CS+US− trials (yellow), and CS− trials (blue) and its 95% confidence limits. FIG. 9D shows the average of decoded arousal state from electrodermal activity (“EDA”) signal across CS+US+ trials (red), CS+US− trials (yellow), and CS− trials (blue) and its 95% confidence limits. The black dash line marks the electric shocks.

FIGS. 10A-10C show the interoceptive awareness and arousal decoders outcomes in the fear conditioning experiment. FIG. 10A represents the outcome of the eye-based IA decoder using a binary observation; FIG. 10B presents the outcome of the EDA-based arousal decoder employing a marked point process observation; and FIG. 10C depicts the EDA-based arousal decoder's outcome employing a binary observation. Sub-panel A shows the physiological signal of interest (i.e., collected from the eye or skin). Sub-panel B shows the applied observation. Sub-panel C shows the state estimates and its 95% confidence limits. (sub-panel D)) The probability of impulse occurrence and its 95% confidence limits. Sub-panel E shows the high interoception index (“HII”)/high arousal index (“HAI”). The red background color corresponds to the CS+US+ trials; the yellow background color stands for CS+US− trials; and the blue one indicates the CS− trials.

FIG. 11 illustrates a computer system for use with certain implementations.

FIG. 12 shows a closed-loop system in which a system for estimation of an IA state from the eye-tracking measurements is used to continuously monitor and shift the IA state.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate generally to systems and methods for estimating interoceptive awareness state using eye-tracking measurements.

Some embodiments relate to a system for detecting eye movements and analyzing information from the user to determine IA state for the use. In one embodiment, a system comprises (1) an eye-tracker, (2) data storage, (3) a processing module comprising of one or more processors connected to the data storage and the eye tracker, (4) an extraction module, (5) an interoceptive awareness state estimator, (6) an interface for communicating with a display (local or remote, such as a wearable or a smart-phone) to show the estimated IA state and enable the communication with the users. FIG. 1 is an illustration of a system for estimation of an interoceptive awareness (“IA”) state from the eye-tracking measurements in accordance with the present disclosure. It should be appreciated that system and methods for tracking the IA state will enable maintaining homeostatic conditions in the body and facilitating self-awareness. This awareness would directly reflect the general mental health (including cognitive and emotional status).

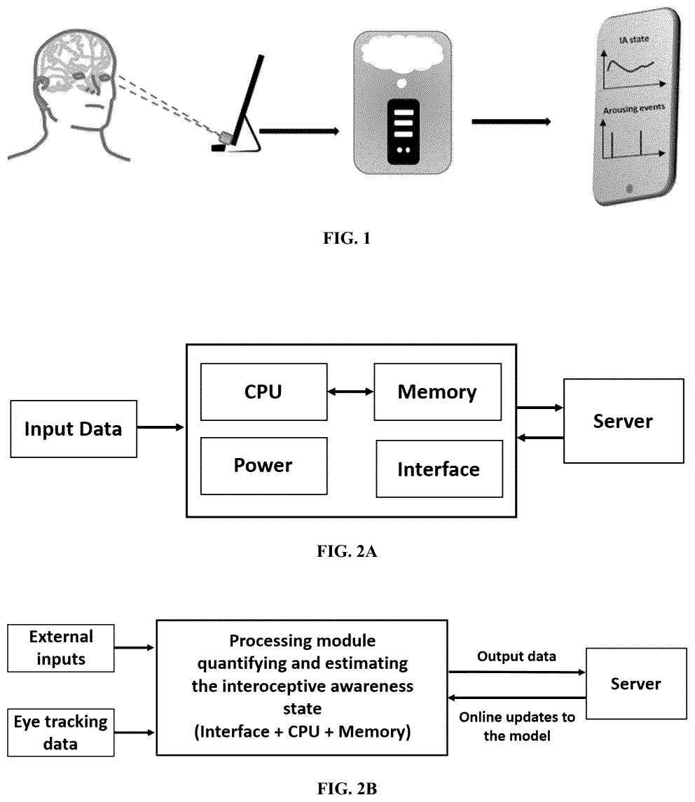

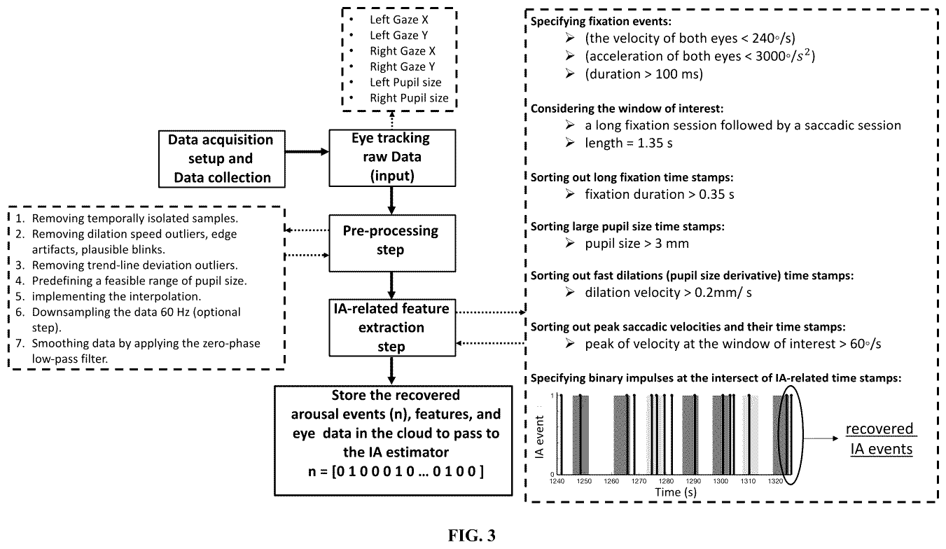

The eye tracker device collects pupil sizes and gaze measurements. In various embodiments, the pupil sizes and gaze measurements are collected from a single eye. In various embodiments, the pupil sizes and gaze measurements are collected from both eyes, and the average of data from both eyes are considered. Systems and methods discussed herein relate to the eye tracker device considering the average of data from both eyes. Systems and methods discussed may also be applied to data collected from a single eye. In one embodiment, the eye tracker device operates a minimum sampling frequency of at least 60 Hz. Both pupillometry and eye gaze are employed to recover the autonomic nervous activities (due to arousing events) from the eye and quantify the underlying interoceptive awareness state, continuously. The data can be stored, such as locally, on a network, in the cloud or the like. FIG. 2A is a block diagram of the system implementation of the system shown in FIG. 1. FIG. 2B is another block diagram of the system implementation of the system shown in FIG. 1.

The eye tracker device may be a calibrated eye tracking system. The calibrated eye tracking system is manually adjusted to estimate a user's eye characteristics and adapts dependent on the characteristics of the user. In one embodiment, the calibrated eye tracking system comprises hardware and systems for manually adjusting and calibrating the eye tracker device. For example, the calibrated eye tracking system may be smart glasses, an augmented reality device, or a virtual reality device. The eye tracker device may be implemented in real-time telemedicine and enable virtual medical assistance by allowing a clinician to virtually assess a patient, such as monitoring a stroke patient's recovery. Other implementations include e-sport and gamification.

The eye tracker device may be a nonspecial consumer system. The nonspecial consumer system may use built-in cameras or sensors to estimate a user's eye characteristics and no manual adjustments or calibrations are applied. For example, the nonspecial consumer system may be a mobile communication device. When compared to the calibrated eye tracking system, because the nonspecial consumer system is not adjusted to each user's eye characteristics, data received from the nonspecial consumer system may be less precise.

The eye tracker device may be a special clinical system. The special clinical system may be used in an exemplary operating environment under managed and ideal guidance. The system and methods discussed herein relate to the eye tracker device configured as the special clinical system. Systems and methods discussed may also be applied to other embodiments of the eye tracker device. The processing module of the system adjusts for the difference in eye tracker devices through a conversion factor that is applied to the data. The extraction module of the system adjusts for the difference in eye tracker devices through thresholds applied to the extraction module.

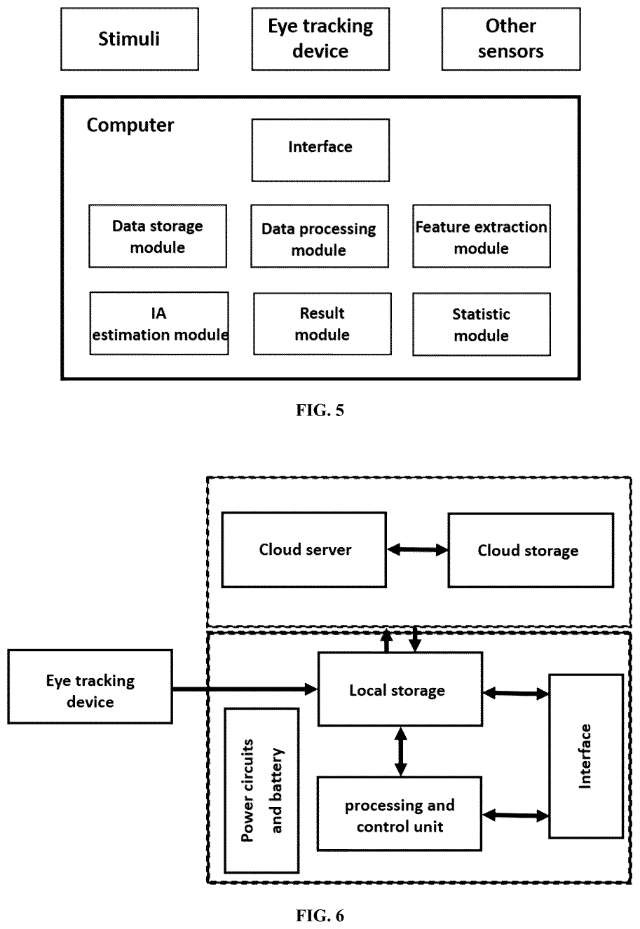

While various operating environments may be used with the system of FIG. 1 or more specifically the system of FIGS. 2A-B, FIG. 5 an illustration of an exemplary operating environment. Similarly, FIG. 6 is a block diagram of a hardware configuration for the exemplary operating environment shown in FIG. 5.

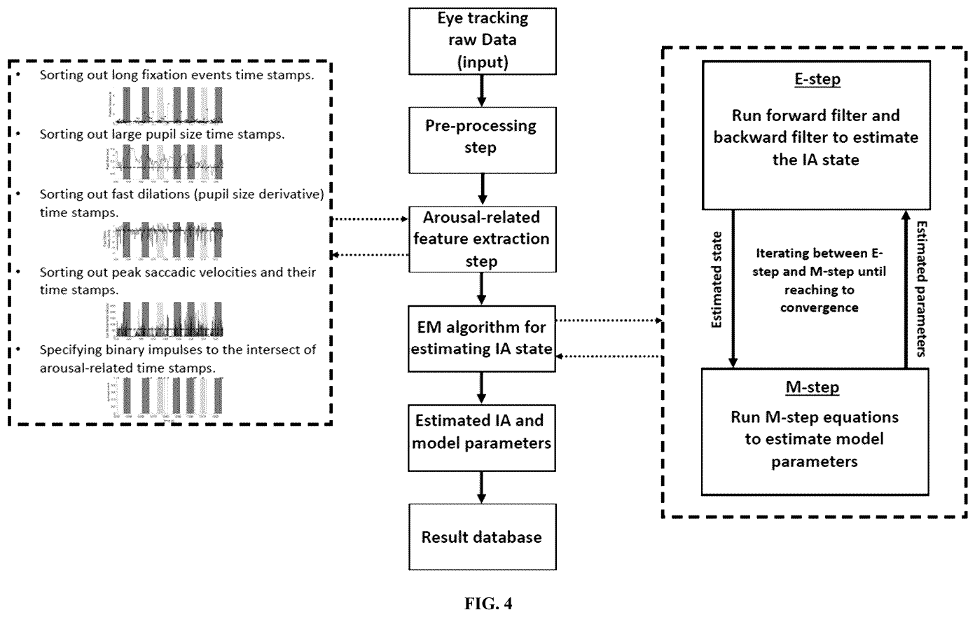

FIG. 3 is an overview of the IA state estimation from the eye movements and pupillometry measurements utilizing a system such as of FIG. 1. FIG. 4 is a flow diagram illustrating a method of estimating an IA state from the eye movements and pupillometry measurements. The initial data acquisition setup and data collection occurs. This may include eye tracking data or may be additional information prior to or after eye tracking data is acquired. The input data, referred to herein as eye tracking data but as noted may include other input data. The input data may be “pre-processed”. While certain data is described in the examples herein, data collection may include array of signals that can be associated with IA. These signals include but are not limited to heart rate, skin conductance, respiration, hormone secretion (e.g., leptin), and even demographic data such as age. Incorporating these signals may provide insight into the estimated IA state and other hidden states. These signals also can be utilized for evaluating decoded IA states such that a hidden state of interest is decoded using these signals separately.

This diagram depicts an overview of the proposed framework given the eye movements and pupillometry measurements as the input of the system, and estimated IA state as the main output, according to an example embodiment. According to the available data, one embodiment of a processing module was utilized to pre-process the information and remove the invalid data points. One embodiment for removing invalid date may be adapted from Kret, Mariska E., and Elio E. Sjak-Shie. “Preprocessing pupil size data: Guidelines and code.” Behavior research methods 51 (2019): 1336-1342, incorporated herein by reference Invalid data is removed using a framework which utilizes the median absolute deviation to detect outliers. The type of eye tracker device used adjusts this invalid data characterization. Specially, temporally isolated samples, dilation speed outliers, edge artifacts, plausible blinks, and trend-line deviation outliers are removed. The interpolation and smoother are implemented to have a smoother signal. The pre-processed data may be stored in local storage to be used. The processing module (including invalid sample removal) may utilize a standard framework in the field that is used to process this type of data. In one embodiment, such as in the examples herein, a framework may be selected is not computationally complex, minimizes information loss, and performs decently in removing invalid data points and smoothing.

The extraction module uses data from the processors to form a binary vector representing decided events. In one embodiment, the goal is to extract the arousal-related features from the pre-processed data. Hence, the extraction module included within the system is an arousal-related feature extraction module and the decided events are arousal-related events. The potential arousal events are recovered through consideration of eye-based variables. These eye-based variables may include the pupil size, pupil size changes, pupillary response, fixation duration, saccadic velocity, and saccade peaks. While the eye-based variables are highly informative on the IA state, and a combination of all of the eye-based variables are used to recover the IA-related neural impulses, other variables such as blink reflex may be applied to form the observation, or each of these variables might be considered solely. However, the combination of all of them may preserve more information on IA and encompass a wide spectrum of the IA. The potential arousal events occurrences are used as the binary observation to estimate the continuous interoceptive awareness state due to the occurrence of arousal events.

In order to estimate the interoceptive awareness state given the binary observation, the interoceptive awareness estimator is utilized. The interoceptive awareness estimator may include the autoregressive (“AR”) model, and the interoceptive awareness state is decoded using the Bayesian filtering approach within the expectation-maximization (“EM”) framework. Those skilled in the art will appreciate that the framework is not limited to AR models; other types of state-space representations can be used. For example, history-dependent models (e.g., autoregressive conditional heteroskedasticity, generalized autoregressive conditional heteroskedasticity, etc.) may be used to incorporate time-varying process noise variance. The embodiments utilizing the AR model with a binary observation do so because it outperforms the other approaches and is simple to implement within the decoder. The system and methods discussed herein relate to the usage of the random walk model, which is a case of the AR model.

The interoceptive awareness estimator may include an estimation module. The estimation module would be the main part that performs the computation to estimate and quantify the hidden interoceptive awareness state. The estimation module utilizes the binary observation to decode the hidden state and the onset of the IA-related neural activity is applied as an estimator input. The estimator input can be adjusted dependent on the binary observations and continuous observations of the eye-based variables. Finally, the result can be presented on the interface and the user can interact within the system.

The following observations can be used, and the estimator can be updated accordingly:
A binary and a continuous observation where the binary observation represents the onset of neural impulses, and the continuous observation can be assigned based on a related continuous measurement such as pupil size. Hence, observation vector can be written as Y^{J}={n₁ and s₁, . . . , n_{J} and s_{J}} where n refers to the occurrence of the impulse and s stands for the continuous variable such as pupil size.
A binary and two continuous observations where the binary observation represents the onset of neural impulses, and the continuous observations can be assigned based on a related continuous measurement such as pupil size and saccadic velocity. Hence, observation vector can be written as Y^{J}={n₁ and s₁ and z₁, . . . , n_{J} and s_{J} and z_{J}} where n refers to the occurrence of the impulse, s stands for the continuous variable such as pupil size, and z indicates the second continuous variable such as saccadic velocity.
A Marked point process observation where the amplitude and the onset of neural impulses are considered as the observation, and the observation vector can be written as Y^{J}={(n₁, r₁), . . . , (n_{J}, r_{J})} where n refers to the occurrence of the impulse, and r indicated the amplitude of the impulse.
A Marked point process observation coupled with a continuous variable where observation vector can be written as Y^{J}={(n₁, r₁) and s1, . . . , (n_{J}, r_{J}) and s_{J}} where n refers to the occurrence of the impulse, r indicated the amplitude of the impulse, and s stands for the continuous variables such as pupil size.

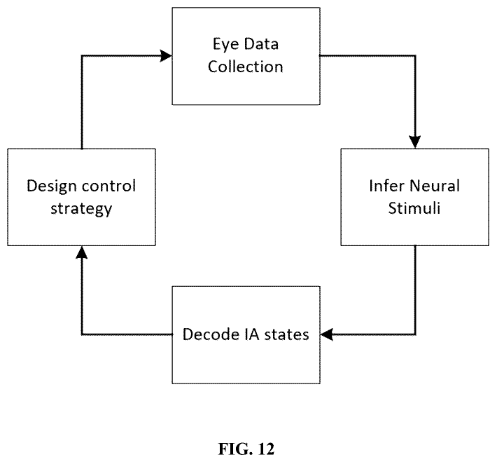

The system and methods for tracking the IA state will enable maintaining allostatic and homeostatic conditions, regulating social behavior, and facilitating emotional awareness. FIG. 12 shows a closed-loop system in which a system for estimation of an IA state from the eye-tracking measurements is used to continuously monitor and shift the IA state. Eye data collection represents the eye tracking data collection using the system. Infer neural stimuli describes the recovery of IA-related neural stimuli using the system. IA-related neural stimuli can be recovered from extracted features such as pupil size, fixation, saccadic velocity, and dilation speed. Decoded IA states depict the decoded continuous trajectory of the IA state. When the decoded continuous trajectory is extremely low, the result may be low arousal condition, and when the decoded continuous trajectory is extremely high, the result may be anxiety and abnormalities in brain-body connection. Design control strategy presents a proper set of interventions that can be used to regulate the decoded IA state.

Continuously tracking the IA state provides an opportunity to set the IA state within a desired range by employing the proper set of interventions. Specifically, the estimated IA state can output a trajectory of the IA state that can assist a user in determining personalized interventions.

An intervention may be a therapeutic intervention. Therapeutic interventions may address mental and health conditions. For example, neuromodulation is a type of therapeutic intervention that can be employed to alter nerve activity. Neuromodulation can be delivered via several approaches, such as electrical stimulation and chemical agents. Nodes of interoceptive pathways can be targeted to shift the IA state in a short period of time. Since the systems and methods for tracking the IA state can reveal the trajectory of the IA state, timing and intensity of a stimulation to nodes of interoceptive pathways can be determined. For example, based on the decoded trajectory of the IA state, individual baseline, and the history of the IA state, the intensity and timing of the stimulation for a stroke patient can be determined.

An intervention may be an everyday-life intervention. Every-day life interventions are affective in moderately alleviating the IA state over a time window. For example, everyday-life interventions may be breathing and mindfulness-based exercises. The systems and methods for tracking the IA state can determine abnormalities in the IA state trajectory and the interface can encourage users to perform breathing and mindfulness-based exercises. Breathing and mindfulness-based exercises can be administered consciously through natural settings. For example, based on the decoded trajectory, individual baseline, and the history of the IA state, the timing of and type of exercise (e.g., breathing exercise) for stress reduction can be determined. In other applications, the timing and dosage of therapy for disorders such as mental disorders can be determined. When compared to therapeutic interventions, everyday-life interventions may be more accessible and easier to implement within everyday life settings.

As the experimental examples below support, the eye is an informative metric of the interoceptive awareness in an arousing condition. In particular, the described processes and systems provide an approach for determining IA state or information relating to IA from observed information for a subject's eye(s). Particularly, pupillometry and eye gaze measurements can be employed together to extract the applicable features, recover the arousal-related events, and build a real-time IA decoder that can be employed within the everyday life settings. Similar to the skin conductance signal, the pupil size signal can be seen as a summation of multiple components. The existence of tonic and phasic components within the pupil size has been established in a typical pupillometry paradigm.

### EXPERIMENTAL EXAMPLES

The studied dataset (publicly available through the Zenodo repository) is described in Y. Xia, F. Melinscak, and D. R. Bach, Saccadic scanpath length: an index for human threat conditioning, Behavior Research Methods 53, 1426 (2021) and R. Amin and R. T. Faghih, Physiological characterization of electrodermal activity enables scalable near real-time autonomic nervous system activation inference, PLOS computational biology 18, e1010275 (2022). The data includes skin conductance responses (“SCR”), electrocardiogram (“ECG”), respiration, pupil size (“PSR”), and gaze coordinates measurements collected from 29 healthy participants (17 females and 12 males) performing a classical (Pavlovian) discriminant delay fear conditioning experiment. There are ten participants (participants 1, 3, 4, 5, 7, 9, 10, 16, 21, and 29) that are excluded from this study due to the lack of unconditioned response (“UR”) to the stimulus (electric shock), not following the task instruction or signal contamination. In this Pavlovian threat conditioning paradigm, two types of visual stimuli are presented as conditioned stimuli (CS+ and CS−), and a 500 ms train of 250 square electric pulses is used as the unconditioned stimulus (“US”). The CS+ is followed by the US half of the time while the CS− is not followed by the US.

The experiment consists of three major blocks. The first two blocks—acquisition phase—are composed of 15 CS+US+ (CS+ with shock), 15 CS+US− (CS+ without shock), and 30 CS− in each block. The last block—extinction phase—comprises 20 CS− and 20 CS+ trials without US occurrence. The order of trials is a random factor in each block. During each trial, the CS is displayed in the first 4 seconds followed by a shock (if the US exists). The inter-trial interval (“ITI”) is randomized to be between 7 and 11 seconds in which 2 seconds of response feedback is presented followed by a fixation cross.

The gaze direction coordinates and pupil sizes were recorded by EyeLink 1000 at a sampling rate of 500 Hz. SCR were collected at a sampling rate of 1000 Hz from the thenar/hypothenar of the non-dominant hand using Biopac system electrodes.

In order to filter out invalid gaze points and pupillometry samples, preprocessing was utilized. For the experiment, three main categories are considered: (1) temporally isolated samples; (2) dilation speed outliers, edge artifacts, and plausible blinks; (3) trend-line deviation outliers. Additionally, specifying 9 mm as the upper, and 1.5 mm as the lower bound of the pupil size, one embodiment predefine a feasible range of pupil size and reject the out-of-range data. Invalid sample removal generates non-equidistant gaps between data. In one embodiment, the data may be interpolated to increase the smoothness. The data may also be down sampled, for example given the average range of saccade duration (20-200 ms), the data may be down sampled to 60 Hz (fₛ=60) to ensure the detection of saccadic movements. The pupil size signal can become smoother by applying the zero-phase low-pass filter with a cutoff frequency of 4 Hz.

The skin conductance raw signal can be considered as a summation of a slow-varying component (i.e., tonic) and a fast-varying component (i.e., phasic). The arousal-related ANS activation can be recovered by modeling the phasic component as the convolution between the neural impulse train and the physiological system response. Thus, to infer the neural impulse train due to ANS activation, a proper deconvolution framework is required: Here, the skin conductance is lowpass filtered at 0.5 Hz and down sampled to 4 Hz with the cut-off frequency of 0.4 Hz. Thereafter, to extract the phasic component, convex optimization approach to electrodermal activity processing (“cvxEDA”) is applied. In order to obtain the ANS activations, signal deconvolution can be performed on the extracted phasic component via a coordinate descent approach.

Given the gaze data in x direction (Xⱼ) and y direction (Yⱼ), and velocity amplitude (vⱼ), and acceleration amplitude (aⱼ) of eye movements, this provides that:

$${v}_{j} = \frac{\sqrt{{( {X}_{j} - {X}_{j - 1} )}^{2} + {( {Y}_{j} - {Y}_{j - 1} )}^{2}}}{{t}_{j} - {t}_{j - 1}} \qquad Eq . 1$$

$${a}_{j} = \frac{{v}_{j} - {v}_{j - 1}}{{t}_{j} - {t}_{j - 1}}$$

where t refers to the time and j stands for the index (j=tfₛ). In the examples described herein, to convert pixels to degrees, based on the experimental setup, 0.024 was used as the conversion factor. The conversion factor is dependent on the configuration of the eye tracker device and the preciseness of the data received from the eye tracker device. The conversion factor can be found based on the distance of the eye tracking device and the eyes. Three conditions were considered based on the nature of this data, for the detection the fixation of the eye: (1) vⱼ<240°/s, (2) aⱼ<3000°/s2, and (3) fixation duration>100 ms. Meeting these three conditions at the same time would lead to detection of fixation. In one embodiment, as the focus is mainly on the fixation onset and duration, the rest of the movements can be treated as saccadic movements. Further, those movements or saccadic movements in general can accounted for by grouping into more specific groups such as noise and movements such as smooth pursuit, microsaccades, and glissade movements.

To form a binary-type observation nⱼ, the information from the pupil size (sⱼ), fixation duration, and saccadic velocity are examined together. The binary observation nⱼ is supposed to be an indicator of arousal-related impulses (arousal events) occurrence in an individual's circuits in response to arousing inputs. As mentioned before, pupil dilation, an increase in the velocity of eye movements, and long fixations all can contribute to expressing the arousing condition. Thus, by setting the thresholds (α₁, α₂, α₃, α₄), and considering window size of interest (Δt→δ), the observation vector n is formed. The window of interest (δ) should start with a fixation movement (τ_{f}) followed by a saccadic movement (τₛ) where the following conditions are satisfied (Δt→δ):

$$Δ t = δ = {τ}_{f} + {τ}_{s} \qquad Eq . 2$$

$${τ}_{f} > {α}_{1} \qquad Eq . 3$$

$${s}_{Δ t \to δ} > {α}_{2} \qquad Eq . 4$$

$$\frac{d s}{d t} > {α}_{3} \qquad Eq . 5$$

$$Max ( {v}_{Δ t \to δ} ) > {α}_{4} \qquad Eq . 6$$

If all the above statements hold, the start of the particular window (i.e., fixation onset) is assumed to be an indicator of the arousal-related impulses where nⱼ=1; otherwise, nⱼ=0. In this study, the thresholds have been set based on the dataset of interest while they are adaptive hyperparameters: α₁=0.35 s, α₂=3 mm, α₃=0.2 mm/s, and α₄=60°/s.

The random walk models, which is a case of the AR model, have been used widely to represent brain hidden states such as arousal and performance. To do so, the IA state zⱼ is modeled as

$${z}_{j} = {z}_{j - 1} + {∈}_{j} \qquad Eq . 7$$

where ∈ⱼ˜ [symbol] (0, σ_{∈}²) is a process noise and σ_{∈}² needs to be determined.

The formed observation (nⱼ) is assumed to follow the Bernoulli-distribution with a probability mass function of pⱼⁿ^{ⱼ}(1−pⱼ)¹⁻ⁿ^{ⱼ}, where pⱼ=P(nⱼ=1). To link the pⱼ to zⱼ, a logit transformation can be applied:

$$log ( \frac{{p}_{j}}{1 - {p}_{j}} ) = λ + {z}_{j} ⟹ {p}_{j} = \frac{1}{1 + {e}^{- ( λ + {z}_{j} )}} \qquad Eq . 8$$

where constant λ can be determined by setting zⱼ≈0 and

$${p}_{0} = \frac{{Σ}_{j = 1}^{j = J} n}{J}$$

(the average probability of nⱼ=1).

$$λ \approx log ( \frac{{p}_{0}}{1 - {p}_{0}} ) \qquad Eq . 9$$

To derive a decoder given the binary-type observation vector, the expectation-maximization (“EM”) framework may be deployed. The EM framework comprises of E-step and M-step where E-step mainly focuses on the state estimation while the M-step is responsible for determining z₀ and σ_{∈}_{ⱼ}² such that they maximize the expected log-likelihood function:

E-step. The E-step includes forward filter and backward smoother. In the forward filtering part, the Bayesian filtering approach is employed to estimate the hidden state (zⱼ). In particular, the fixed values of z₀ and σ_{∈}_{ⱼ}² are considered so as to estimate the hidden state.

### Predict:

$${z}_{j \vert j - 1} = {z}_{j - 1 \vert j - 1} \qquad Eq . 10$$

$${σ}_{j \vert j - 1}^{2} = {σ}_{j - 1 \vert j - 1}^{2} + {σ}_{∈}^{2} \qquad Eq . 11$$

### Update:

$${z}_{j \vert j} = {z}_{j \vert j - 1} + {σ}_{j \vert j - 1}^{2} [ {n}_{j} - \frac{1}{1 + {e}^{- ( λ + {z}_{j \vert j} )}} ] \qquad Eq . 12$$

$${σ}_{j \vert j}^{2} = {[ \frac{1}{{σ}_{j \vert j - 1}^{2}} + \frac{{e}^{λ + {z}_{j \vert j}}}{1 + {e}^{λ + {z}_{j \vert j}}} ]}^{- I} \qquad Eq . 13$$

By reversing the direction, z_{j|J} and σ_{j|J}² denote the smoothed state and variance, respectively.

$${A}_{j} = \frac{{σ}_{j \vert j}^{2}}{{σ}_{j + 1 \vert j}^{2}} \qquad Eq . 14$$

$${z}_{j \vert J} = {z}_{j \vert j} + {A}_{j} ( {z}_{j + 1 \vert J} + {z}_{j + 1 \vert j} ) \qquad Eq . 15$$

$${σ}_{j \vert J}^{2} = {σ}_{j \vert j}^{2} + {A}_{j}^{2} ( {σ}_{j + 1 \vert J}^{2} - {σ}_{j + 1 \vert j}^{2} ) \qquad Eq . 16$$

M-step. In the M-step, unknown terms (σ_{∈}², z₀) such that they maximize the expected value of the following log-likelihood function (Q):

$$Q = \frac{- J}{2} log ( 2 π {σ}_{∈}^{2} ) - {\sum }_{j = 1}^{J} \frac{𝔼 [ {( {z}_{j} - {z}_{j - 1} )}^{2} ]}{2 {σ}_{∈}^{2}} \qquad Eq . 17$$

### Hence:

$${σ}_{∈}^{2} = \frac{2}{J + 1} [ {\sum }_{j = 2}^{J} ( {σ}_{j \vert J}^{2} + {z}_{j \vert J}^{2} ) - {\sum }_{j = 2}^{J} ( {A}_{j} {Σ}_{j \vert J} {z}_{j \vert J} {z}_{j - 1 \vert J} ) ] + \frac{1}{J + 1} [ \frac{3}{2} {z}_{1 \vert J}^{2} + 2 {σ}_{1 \vert J}^{2} - ( {σ}_{J \vert J}^{2} + {z}_{J \vert J}^{2} ) ] \qquad Eq . 18$$

$${z}_{0} = \frac{1}{2} {z}_{1 \vert J} \qquad Eq . 19$$

The algorithm iterates between the E-step and the M-step until satisfying the convergence criteria. We can apply similar framework and use the recovered ANS activation as the binary observation to decode the hidden arousal state (x).

The averages of estimated IA and arousal states using binary filters and associated with CS+US+ and US− are presented in Table II. For the average of estimated states, a one tail t-test is performed. The significant levels are set at P≤0.05 and the t-value and p-value are reported given the alternative hypothesis, ha: states at CS+US+>states at CS− (hₐ: CS+US+>CS−). Based on the frequency of the EDA signal and the eye-tracking measurements, the number of data points associated with EDA-based decoder at each trial is 25 while the number of data points associated with eye-based decoder is 376 in the performed ERP-like study.

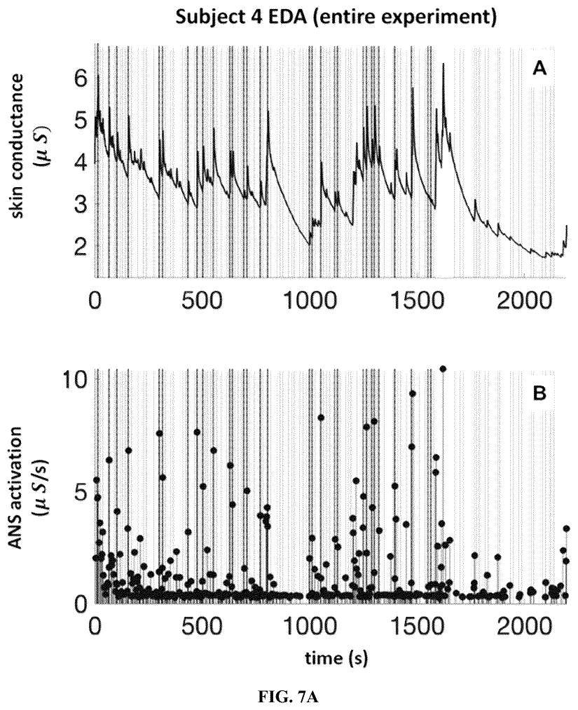

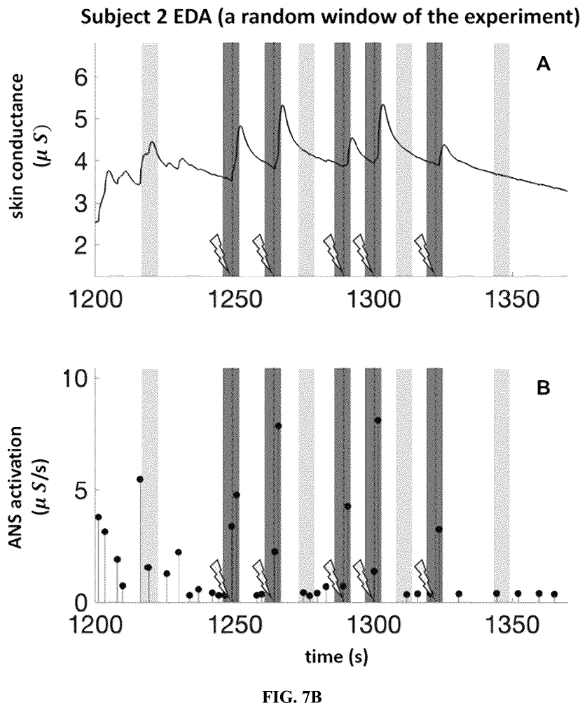

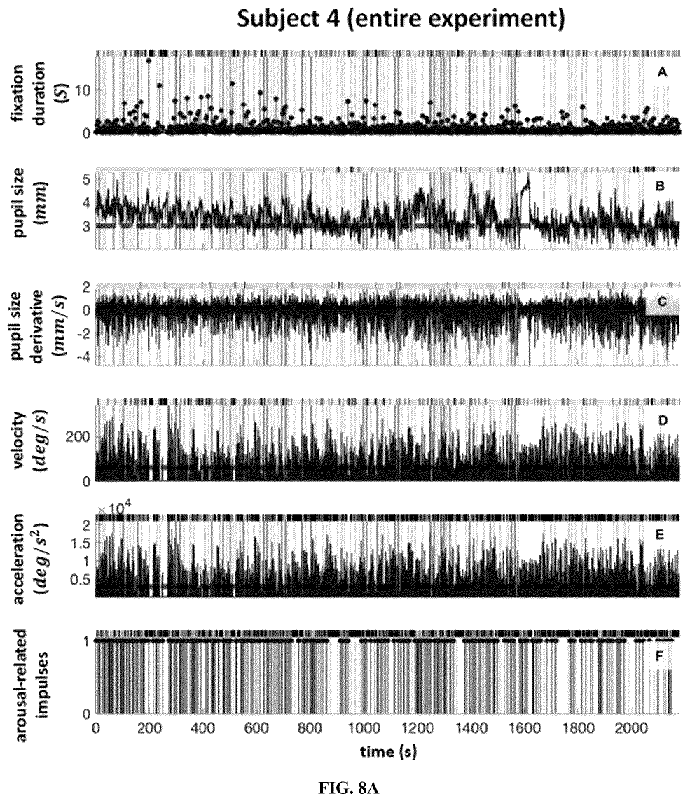

FIGS. 7A-7B present the recorded skin conductance signal (subplot A) and recovered arousal-related ANS activation using deconvolution framework (subplot B) for one subject. Participants without the unconditioned response (“UR”) to the stimulus (electric shock) in their skin conductance signal can be detected and excluded from the study. A random window of the experiment shown in FIG. 7B depicts the skin conductance response to three types of trials: (1) trials with CS+US+ stimulus (red) where electric shock exists; (2) trials with CS+US− stimulus (yellow) where electric shock is absent; (3) trials with CS− stimulus (blue).

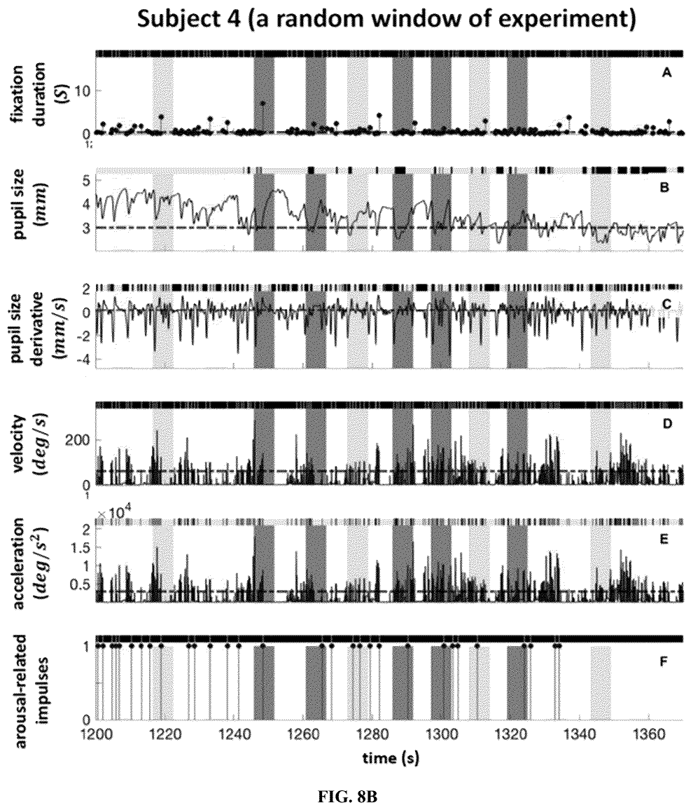

The arousing feature extraction steps from the eye are explained and visualized in FIGS. 8A-8B. To form the binary vector of arousal-related events, fixation duration (first subplot), pupil size (second subplot), pupil dilation (third subplot), velocity (fourth subplot), and acceleration (fifth subplot) of the eye are considered. The intersection of all time stamps that meet the conditions and pass the specified thresholds (covered in method section) would generate a sparse binary impulse and stands for the recovered arousal-related neural activities (sixth subplot).

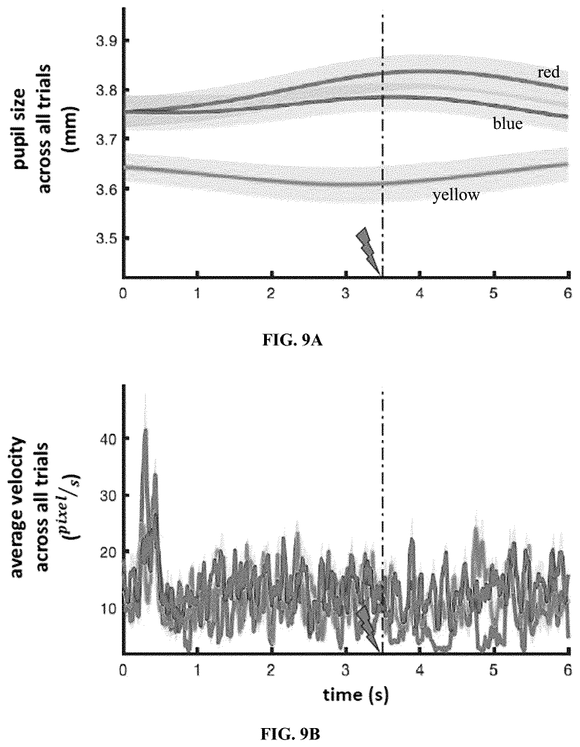

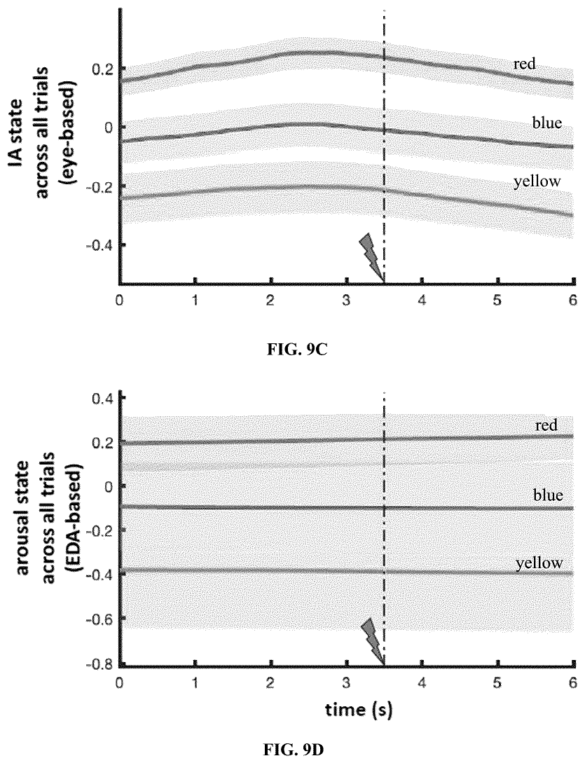

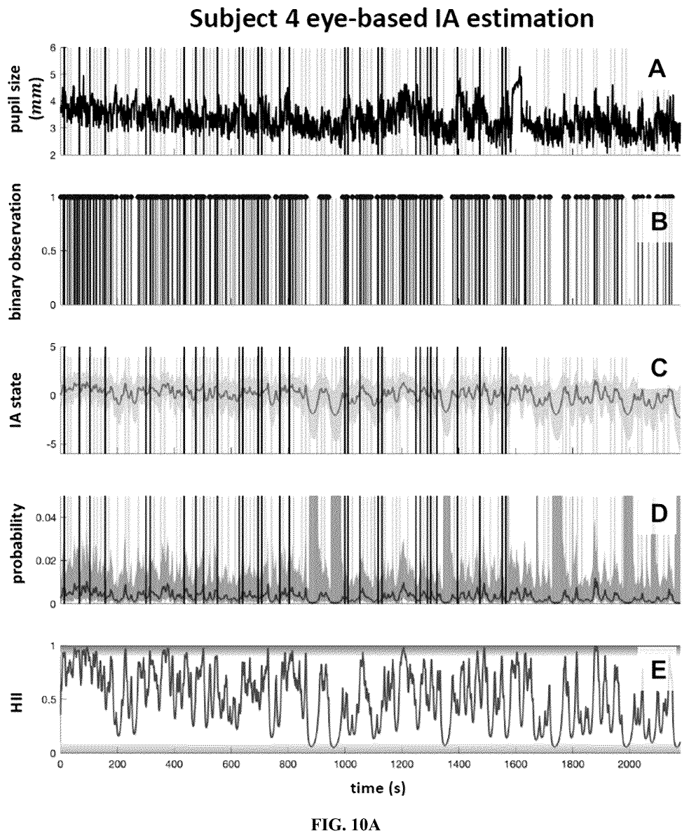

The average of available signals over the experiment's trials was analyzed with respect to each type of stimuli: CS+US+ (red), CS+US− (yellow), and US− (blue). The purpose of this event-related potential like (ERP-like) analysis is to characterize the signal's trend related to the event of interest (presented type of stimuli) within the epochs. FIGS. 9A-9D demonstrate a stimulus-wise perspective of the average pupil size signal, the average velocity across all trials, estimated IA states using a binary observation from eye-tracking and estimated arousal state using a binary observation from the EDA signal, respectively (subplots A to D). Also, in order to underline the differences between the employed features, the average fixation duration (τ_{f}), pupil size(s), and the peak of the saccadic velocity at each type of stimuli are represented in Table I.

| Original | | CS + US+ | | | CS− | | |
|---|---|---|---|---|---|---|---|
| Participant | Subject | d̅_{f} | s̅ | Max(v) | τ̅_{f} | s̅ | Max(v) |
| 2 | 1 | 0.493 | 2.943 | 19.249 | 0.034 | 2.865 | 27.495 |
| 6 | 2 | 0.119 | 3.490 | 15.531 | 0.047 | 3.33 | 13.411 |
| 8 | 3 | 0.100 | 4.154 | 7.900 | 0.048 | 4.158 | 8.429 |
| 11 | 4 | 0.037 | 3.804 | 39.369 | 0.015 | 3.761 | 23.120 |
| 12 | 5 | 0.106 | 3.889 | 9.196 | 0.046 | 4.005 | 7.982 |
| 13 | 6 | 0.072 | 3.944 | 36.605 | 0.020 | 3.885 | 17.819 |
| 14 | 7 | 0.169 | 3.200 | 11.665 | 0.104 | 3.174 | 6.275 |
| 15 | 8 | 0.048 | 2.704 | 8.112 | 0.029 | 2.707 | 6.312 |
| 17 | 9 | 0.110 | 2.174 | 12.619 | 0.051 | 2.168 | 17.616 |
| 18 | 10 | 0.034 | 3.279 | 34.105 | 0.015 | 3.370 | 27.449 |
| 19 | 11 | 0.059 | 3.127 | 25.713 | 0.035 | 3.033 | 16.298 |
| 20 | 12 | 0.044 | 3.786 | 9.016 | 0.022 | 3.831 | 12.474 |
| 22 | 13 | 0.073 | 2.829 | 28.213 | 0.036 | 2.812 | 30.440 |
| 23 | 14 | 0.063 | 2.974 | 7.703 | 0.044 | 2.988 | 7.857 |
| 24 | 15 | 0.049 | 3.039 | 16.853 | 0.023 | 3.049 | 15.025 |
| 25 | 16 | 0.034 | 3.350 | 30.440 | 0.015 | 3.345 | 28.806 |
| 26 | 17 | 0.108 | 2.695 | 12.064 | 0.067 | 2.644 | 8.669 |
| 27 | 18 | 0.063 | 3.105 | 14.360 | 0.037 | 2.950 | 13.373 |
| 28 | 19 | 0.061 | 3.144 | 9.851 | 0.027 | 3.114 | 7.868 |

Table I shows eye-tracking features across the trials. This table includes the specified features during CS+US+, and CS− trials of the experiment which highlights the impact of the electric shock (US).

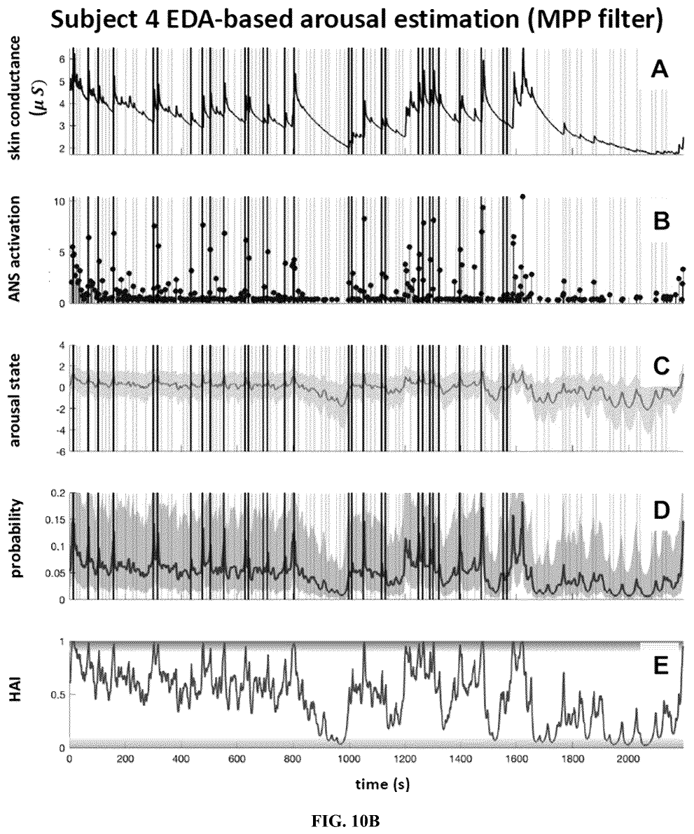

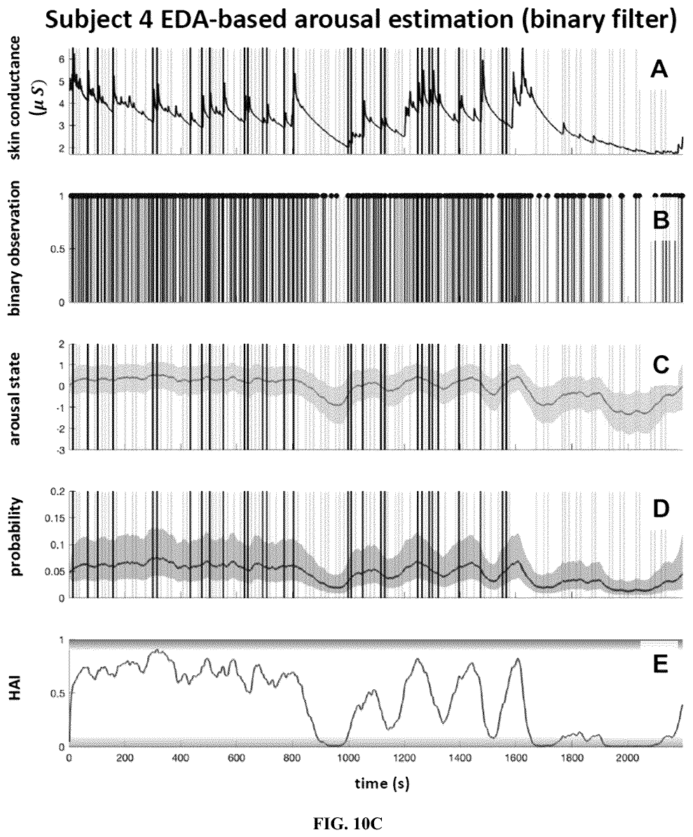

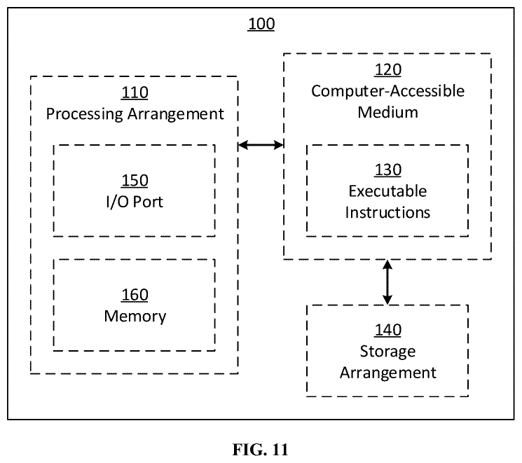

Given the available observations, the hidden IA and arousal states are decoded. FIGS. 10A-10C demonstrate the outcomes of decoders. Three different decoders are studied: FIG. 10A shows the binary-type IA decoder's outcomes where the binary observation is constructed from the eye-tracking; FIG. 10B represents a marked point process (“MPP”) EDA-based arousal decoder where the applied observation vector consists of the recovered ANS activation from the EDA signal, and their corresponding amplitudes; and FIG. 10C represents the binary arousal decoder derived from the EDA-based binary observation. Subplot B of each sub-figure denotes the type of applied observation (binary or MPP), subplot C of each sub-figure indicates a decoded state, and subplot D of each sub-figure presents the probability of observing arousal-related neural firing (arousal events). Similar to the previous work on HAI, the interoception level of the subject is generalized during the whole experiment via the HII. This type of generalization for binary impulse has been introduced to denote that the occurrence of a binary impulse is more than just by chance, and it can be derived from Pr(zⱼ>median(z_{1:J})). Also, the averages of estimated IA and arousal states using binary decoders associated with CS+US+ and US− are reported in Table II.

| | | | | | t-values: | | p-values: | |
|---|---|---|---|---|---|---|---|---|
| | | | | | CS + US+ > CS− | | CS + US+ > CS− | |
| | CS + US+ | | CS− | | t-value | t-value | p-value | p-value |
| Subject | x̅_{eda} | z̅_{eye} | x̅_{eda} | z̅_{eye} | (EDA) | (eye) | (EDA) | (eye) |
| 1 | 0.096 | −0.012 | −0.034 | 0.066 | 540.037 | −346.119 | <0.0001 | >0.99 |
| 2 | 0.123 | 0.154 | −0.181 | −0.225 | 279.167 | 539.664 | <0.0001 | <0.0001 |
| 3 | −0.102 | 0.086 | 0.050 | −0.012 | −477.001 | −102.763 | >0.99 | >0.99 |
| 4 | 0.2289 | 0.207 | −0.103 | −0.025 | 201.322 | 13.637 | <0.0001 | <0.0001 |
| 5 | −0.120 | −0.036 | 0.019 | 0.059 | −146.163 | −203.108 | >0.99 | >0.99 |
| 6 | −0.016 | −0.104 | 0.033 | 0.056 | −101.088 | −120.108 | >0.99 | >0.99 |
| 7 | 0.067 | 0.005 | −0.006 | −0.160 | 211.463 | 471.225 | <0.0001 | <0.0001 |
| 8 | −0.113 | −2.278 | −0.003 | −2.478 | −151.670 | 887.248 | >0.99 | <0.0001 |
| 9 | −0.229 | −1.628 | −0.090 | −1.664 | −98.066 | 338.557 | >0.99 | <0.0001 |
| 10 | −0.091 | 0.0894 | −0.016 | −0.071 | −121.705 | 262.119 | >0.99 | <0.0001 |
| 11 | −0.064 | 0.133 | 0.008 | −0.265 | −125.870 | 1276.433 | >0.99 | <0.0001 |
| 12 | −0.083 | −0.022 | −0.054 | 0.1 | −41.568 | 106.287 | >0.99 | >0.99 |
| 13 | 0.102 | −0.273 | −0.035 | −0.166 | 182.440 | −393.680 | <0.0001 | >0.99 |
| 14 | 0.087 | 0.380 | −0.010 | −0.705 | 123.947 | 1168.695 | <0.0001 | <0.0001 |
| 15 | −0.204 | 0.171 | 0.007 | −0.120 | −442.773 | 335.838 | >0.99 | <0.0001 |
| 16 | −0.093 | 0.087 | 0.002 | −0.004 | −782.779 | 224.646 | >0.99 | <0.0001 |
| 17 | −0.011 | −0.101 | −0.009 | −0.207 | −9.452 | 246.759 | >0.99 | <0.0001 |
| 18 | −0.035 | −0.347 | −0.001 | −0.211 | −137.451 | −178.224 | >0.99 | >0.99 |
| 19 | 0.158 | 0.270 | −0.026 | −0.186 | 261.326 | 1198.573 | <0.0001 | <0.0001 |

Table II shows the average of decoded IA and arousal states using binary decoders across the CS+US+ and CS− trials. This table demonstrates the average of decoded IA and arousal states across CS+US+ and CS− trials of the experiment for both EDA-based and eye-based binary decoders. The EDA-based decoder produces 25 arousal data points at each trial while the eye-based decoder considers 376 IA data points at each trial in the performed ERP-like study. The one tail t-test is performed. For the EDA-based arousal state (x_{eda}), the degree of freedom is 48 while for the eye-based IA state (z_{eye}), the degree of freedom is 750; the t-values and p-values are reported above.

The main objective of this research is to investigate the features of the eye to decode the IA state of the brain. Although several neural pathways are likely to be involved in experiencing and expressing interoceptive sensing such as arousal, facial muscle and eye movements appear to be involved in the expressions of arousal. Here, with some caveats, a comprehensive pipeline is provided such that it accounts for pupillometry and eye gaze to decode the underlying IA state. To evaluate the outcome of the eye-based decoder, the EDA signal is employed to decode and compare the arousal alongside the eye-based IA state. Since the studied fear conditioning experiment has three major types of stimuli (i.e., CS+US+, CS+US−, and CS−), one approach is to detect the impacts of each stimulus. This evaluation is inspired by the event-related potential (“ERP”) technique with the EEG data.

In FIGS. 7A-7B, the sharp increases of the skin conductance signal in response to the electric shock are observable which implies the informativeness of the skin conductance data regarding the arousal condition. The proposed deconvolution framework in Amin & Faghih, “Physiological characterization of electrodermal activity enables scalable near real-time autonomic nervous system activation inference,” PLOS computational biology 18, e1010275 (2022) can be used to recover the associated ANS activities such that in most of the CS+US+ trials, there is a decent trend of high-value impulses observable by the unaided eye. Additionally, there is an agreement between the EDA-based arousal estimation in this study and findings related to arousal in Wickramasuriya & Faghih, “A mixed filter algorithm for sympathetic arousal tracking from skin conductance and heart rate measurements in Pavlovian fear conditioning,” PloS one 15, e0231659 (2020); in both Pavlovian experiments, the CS+US+ trials reveal the highest state level (FIG. 9D).

Considering pupillometry and eye gaze, FIGS. 8A-8B depicts the feature extraction process and arousal event inference. In one embodiment, the objective is to form the sets of bins from the eye features and sparsify them to build an observation vector which reveals the underlying neural activities in response to the arousing condition. Explicitly, the first feature to be evaluated is the fixation events' occurrence and duration (subplot A). The fixation duration varies from several milliseconds to several seconds. These longer fixation duration events are of particular interest. The applied threshold for sorting out the bins depends on the nature of the experiment and individual conditions. According to the subject's baseline, the fixation duration threshold is set to 0.35 seconds. The pupil size is the next informative feature to be considered. Normally, the pupil size varies from 2 mm to 8 mm, and the lighting condition plays a key role in determining the pupil size. In subplot B of FIGS. 8A-8B, the pupil size takes values in the range of 2-6 mm for this particular subject. Based on the subject's baseline, the threshold is set to 3 mm. As depicted in subplot B of FIGS. 8A-8B, there is an increase in pupil size such that it passes the 3 mm threshold in most of the CS+US+ trials (red regions). This increase in pupil size is consistent with the pupil dilation in response to experiencing a high arousal condition and receiving shock; this behavior inspired us to further investigate the derivative of pupil size throughout the experiment. Hence, based on the subject's data, bins are formed where the derivative of pupil size passes 0.2 mm/s threshold (subplot C). The velocity of the eye movement appears to be informative regarding the arousal condition. According to subplot E of FIGS. 8A-8B, the sharp increase in velocity can be detected in response to the stimuli while during the inter-trial interval (“ITI”), the velocity decreases. The applied threshold for detecting the sharp increases in velocity is 60°/s. The acceleration amplitude is considered in the fixation detection step where the acceleration amplitude is aⱼ<3000°/s2. Eventually, the observation vector is formed (subplot F of FIGS. 8A-8B) based on the applied steps.

ERP-like perspective of the data is shown in FIGS. 9A-9D. Previously, it has been shown that arousal levels are high in response to the CS+US+. There is an agreement between the EDA-based and eye-based estimates such that the highest state levels occur at the CS+US+ stimuli. Additionally, the higher values of pupil size and IA state can be detected during the CS+US+ trials. Another point to consider is that the highest and lowest average velocity values correspond to the CS+US+. In one embodiment, the velocity values close to zero are interpreted as the moments of fixation while the higher velocity stands for the saccadic movements.

Subplots of FIGS. 10A-10C indicate in turn the eye-based IA estimation from binary observation, the EDA-based arousal estimation from MPP observation, and the EDA-based arousal estimation from binary observation. By comparing the EDA-based state estimation results, it can be seen that states and HAI from the MPP decoder have higher variations compared to the states and HAI derived from the binary decoder which is smoother. Possibly, accounting for the amplitude of impulses would induce the noise. Although the binary observation is fed to the eye-based decoder, the decoded IA from the eye has a noticeable fluctuation. The smoothness of the estimated state can be related to the sparsity level of the observation vector, and the noise dynamic of the state model. Hence, thresholding and processing steps of the eye-tracking data can play a crucial role in this paradigm.

Described herein are methods and systems for identifying the informative features of the eye-tracking signal. In one embodiment, a pre-processing approach is used. However, in other embodiments, other pre-processing, including more complex approaches, as well as smoothing frameworks that use the Hampel and Savitzky-Golay filters, can be utilized and may impact the outcome. Additionally, by comparing the skin conductance and the pupil size signals, one may characterize the pupil size signal as a summation of multiple components. Hence, there might be a need for considering an informative component of the pupil size signal, solely.

Given the data in Table II, the experiments considered the potential impact of the electric shock (US), for resulting in three different scenarios:
1. For 10 subjects (subjects 2, 3, 4, 5, 6, 7, 12, 14, 18, 19), there is an agreement between the EDA-based and eye-based estimations in terms of the order of state levels at CS+US+ and CS− trials.
2. For 7 subjects (subjects 8, 9, 10, 11, 15, 16, 17), there is a disagreement between the EDA-based and eye-based estimations in terms of the order of state levels at CS+US+ and CS− trials. While the eye-based decoder estimates higher average IA for CS+US+ trials compared to CS− trials, the EDA-based arousal decoder results show an opposite pattern.
3. For 2 subjects (subjects 1, 13), the EDA-based decoder estimates higher average arousal among CS+US+ trials compared to CS− trials while the eye-based IA decoder reveals an opposite trend for IA state.

In view of the experimental results, the system and methods were able to provide IA information when in an arousing condition.

### Definitions

As used herein, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

As used herein, the terms “coupled,” “connected,” and the like mean the joining of two additional intermediate members being integrally formed as a single unitary body with one another or with the two members or the two members and any additional intermediate members being attached to one another.

As shown in FIG. 11, e.g., a computer-accessible medium 120 (e.g., as described herein, storage members directly or indirectly to one another. Such joining may be stationary (e.g., permanent) or moveable (e.g., removable or releasable). Such joining may be achieved with the two members or the two members and any device such as a hard disk, floppy disk, memory stick, CD-ROM, RAM, ROM, etc., or a collection thereof) can be provided (e.g., in communication with the processing arrangement 110). The computer-accessible medium 120 may be a non-transitory computer-accessible medium. The computer-accessible medium 120 can contain executable instructions 130 thereon. In addition, or alternatively, a storage arrangement 140 can be provided separately from the computer-accessible medium 120, which can provide the instructions to the processing arrangement 110 so as to configure the processing arrangement to execute certain exemplary procedures, processes and methods, as described herein, for example. The instructions may include a plurality of sets of instructions.

System 100 may also include a display or output device, an input device such as a keyboard, mouse, touch screen or other input device, and may be connected to additional systems via a logical network. Many of the embodiments described herein may be practiced in a networked environment using logical connections to one or more remote computers having processors. Logical connections may include a local area network (“LAN”) and a wide area network (“WAN”) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule-based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

What is claimed is:

1. A system for continuously estimating interoceptive awareness comprising:
an eye tracker device, the eye tracker device collecting eye-tracking data comprising pupillometry measurements and gaze measurements;
a data storage, the eye-tracking data stored in the data storage;
a processing module comprising of one or more processors, the processors connected to the eye tracker device and the data storage to output processed data;
an extraction module, the extraction module using the processed data to form a binary vector, the binary vector representing decided events;
an interoceptive awareness state estimator, the interoceptive awareness state estimator using the binary vector and the eye-tracking data in the data storage to produce an estimated interoceptive awareness state; and
an interface, the interface interacting with a display, the display showing the estimated interoceptive awareness state.

2. The system of claim 1, wherein the eye tracker device operates a minimum sampling frequency of at least 60 Hz.

3. The system of claim 1, wherein the eye tracker device is a calibrated eye tracking system, the calibrated eye tracking system comprising of hardware for manually adjusting and calibrating the eye tracker device.

4. The system of claim 3, wherein the calibrated eye tracking system is an augmented reality device or a virtual reality device.

5. The system of claim 1, wherein the eye tracker device is a nonspecial consumer system, the nonspecial consumer system comprising cameras or sensors.

6. The system of claim 5, wherein the nonspecial consumer system is a mobile communication device.

7. The system of claim 1, wherein the processing module applies a conversion factor to the eye-tracking data based on the preciseness of the eye tracker device used.

8. The system of claim 1, further comprising an external input data, the external input data comprising an array of signals, the array of signals representing factors associated with the interoceptive awareness, wherein the external input data is stored in the data storage and processed with the eye-tracking data.

9. The system of claim 1, wherein the extraction module is an arousal-related feature extraction module, the arousal-related feature extraction module forming the binary vector, the binary vector representing arousal-related events.

10. The system of claim 1, wherein the processing module is configured to remove invalid eye-tracking data points and the eye-tracking data is passed through a low-pass filter.

11. The system of claim 1, wherein the interoceptive awareness state estimator comprises:
an autoregressive model;
an expectation-maximization framework, the expectation-maximization framework comprising:
an E-step, the E-step utilizing a Bayesian filtering approach, and
an M-step; and
an estimation module.

12. A method for continuously estimating interoceptive awareness comprising:
collecting, by an eye tracker device, eye-tracking data, the eye-tracking data comprising of pupillometry measurements and gaze measurements;
storing, by a data storage, the eye-tracking data;
processing, by a processing module comprising of one or more processors connected to the eye tracker device and the data storage, the eye-tracking data, resulting in processed data;
forming, by an extraction module using the processed data, a binary vector, the binary vector representing decided events;
producing, by an interoceptive awareness state estimator using the binary vector and the eye-tracking data, an estimated interoceptive awareness state; and
displaying, through an interface interacting with a display, the estimated interoceptive awareness state to a user.

13. The method of claim 12, wherein the eye tracker device operates a minimum sampling frequency of at least 60 Hz.

14. The method of claim 12, wherein the eye tracker device is a calibrated eye tracking system, the calibrated eye tracking system comprising of hardware for manually adjusting and calibrating the eye tracker device.

15. The method of claim 12, wherein the eye tracker device is a nonspecial consumer system, the nonspecial consumer system comprising cameras or sensors.

16. The method of claim 12, wherein the method further comprises of using an external input data, the external input data comprising of an array of signals, the array of signals representing factors associated with interoceptive awareness, and the external input data can be collected before or directly after the eye-tracking data is collected.

17. The method of claim 12, wherein the extraction module is an arousal-related feature extraction module, the arousal-related feature extraction module forming the binary vector, the binary vector representing arousal-related events through a process comprising of:
recording eye-based features over time, eye-based features comprising:
fixation duration,
pupil size,
pupil size derivative,
velocity, and
acceleration;
comparing, for each eye-based feature, the eye-based feature at every time to a threshold value; and
developing, for times when the eye-based features reach the threshold value, a binary impulse.

18. The method of claim 12, wherein the processing module is configured to remove invalid eye-tracking data points and the eye-tracking data is passed through a zero-phase low-pass filter.

19. The method of claim 12, wherein the interoceptive awareness state estimator produces the estimated interoceptive awareness state through a process comprising of:
an autoregressive model;
an expectation-maximization framework, the expectation-maximization framework comprising:
an E-step, the E-step utilizing a Bayesian filtering approach, and
an M-step; and
an estimation module.

20. The method of claim 19, wherein the estimation module is configured to utilize the binary vector and continuous observations of one or more eye-based features to update an output of the estimated interoceptive awareness state.
